(19) United States
(12) Patent Application Publication
(10) Pub. No.: US 2015/0032034 A1
(43) Pub. Date: Jan. 29, 2015

(54) APPARATUS AND METHOD FOR QUANTIFYING STABILITY OF THE KNEE

(71) Applicant: THE REGENTS OF THE UNIVERSITY OF CALIFORNIA, Oakland, CA (US)

(72) Inventors: Frank A. Petrigliano, Santa Monica, CA (US); Henrik Borgstrom, Los Angeles, CA (US); Keith Markolf, Pacific Palisades, CA (US); David R. McAllister, Los Angeles, CA (US); William Kaiser, Los Angeles, CA (US); Mahdi Ashktorab, San Jose, CA (US)

(73) Assignee: THE REGENTS OF THE UNIVERSITY OF CALIFORNIA, Oakland, CA (US)

(21) Appl. No.: 14/455,709

(22) Filed: Aug. 8, 2014

Related U.S. Application Data
(63) Continuation of application No. PCT/US2013/026229, filed on Feb. 14, 2013.
(60) Provisional application No. 61/598,895, filed on Feb. 14, 2012.

(51) Int. Cl. A61B 5/00; A61B 5/11

(52) U.S. Cl. CPC A61B 5/4585; A61B 5/1122; A61B 5/7242; A61B 5/6828; A61B 5/7282
USPC 600/595

(57) ABSTRACT

A wireless motion sensor platform comprising MEMS inertial sensors and accompanying software for classification of diverse motion characteristics and kinematics of patient anatomy at high resolution. The sensor platform comprises a low-cost, compact, and low-weight device that can be applied to a patient's upper and/or lower leg during a knee examination to measure acceleration along three axes as well as rotations about these axes.

### CROSS-REFERENCE TO RELATED APPLICATIONS

This application is a 35 U.S.C. §111(a) continuation of PCT international application number PCT/US2013/026229 filed on Feb. 14, 2013, incorporated herein by reference in its entirety, which claims priority to, and the benefit of, U.S. provisional patent application Ser. No. 61/598,895 filed on Feb. 14, 2012, incorporated herein by reference in its entirety. Priority is claimed to each of the foregoing applications.

The above-referenced PCT international application was published as PCT International Publication No. WO 2013/123263 on Aug. 22, 2013 which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to systems for evaluating kinematics of skeletal joints, and more particularly to kinematic evaluation of the knee.

2. Description of Related Art

Rupture of the anterior cruciate liagment (ACL) is one of most common ligament injuries of the knee and results in 250,000 ACL reconstructions performed nationally each year. The ACL is the most important stabilizer of the knee and is necessary for sports that require pivoting. In some patients, it is necessary for low-impact activities such as walking. When injured, its absence often leads to episodic instability and may necessitate activity modifications to avoid these symptoms. There is evidence to suggest that if left untreated, progressive injury to knee cartilage may ensue. Thus, the management of ACL injuries is directed at restoring knee stability to prevent these potential sequelae.

Contemporary management of ACL insufficiency involves replacing the injured ligament with a graft that aims to restore both translational (anterior-to-posterior) and rotational (axial) stability to the knee. Contemporary measurements of knee stability are generally predicated on either a subjective physical exam or the KT-1000 arthrometer. The Lachman test (quantified by the KT-1000 arthometer), which measures anteroposterior translational stability, was the gold standard method for determining the efficacy of ACL reconstruction. Unfortunately, this method is limited to a uniplanar analysis of knee laxity that does not correlate with subjective symptoms of instability.

Recently, dynamic rotational stability testing of the knee has drawn a great deal of attention from both clinicians and researchers as the proposed standard for assessing knee stability in the ACL-deficient and ACL-reconstructed state. The impetus for the use of these tests is predicated on clinical studies that suggest that subjective symptoms and patient satisfaction are more closely correlated with the presence of dynamic rotational instability measured via a ‘pivot shift’ exam rather than increased laxity measured by translational stability exams. While the pivot shift exam is widely accepted as a measure of rotational stability, there are currently no objective means of quantifying this exam.

There have been motion tracking devices that have been adapted to the purpose of measuring multiplanar or rotational knee stability. However, these devices are large, fixed position constructs that would not be amenable to use in the clinic, and therefore, are impractical. One example is the use of computer aided navigation to track multiplanar knee instability in the operating room. However, to utilize this device, large 5 mm pins must be drilled into the tibia and femur, and optical tracking reflectors are attached to the pins. While this method is accurate in tracking multiplanar instability, it is not practical to use in the outpatient setting, and is costly and time consuming in the operating room.

More recently, other investigators have proposed the use of small accelerometers to characterize movements about the knee. These devices have demonstrated differences in acceleration patterns in the ACL-intact and ACL-deficient knee; however, they are not designed to measure rotational stability of the knee or provide absolute values of rotational stability. Consequently, the value of the data provided by these instruments is of limited clinical value.

### BRIEF SUMMARY OF THE INVENTION

An aspect of the present invention is a wireless motion sensor platform built around MEMS inertial sensors and accompanying software that permits classification of diverse motion characteristics and kinematics of patient anatomy at high resolution. In one embodiment, the sensor platform comprises a low-cost, compact, and low-weight device that can be applied to a patient's upper and/or lower leg during a knee examination to measure acceleration along three axes as well as rotations about these axes.

While the figures and discription below are primarily directed to kinematic evaluation of the knee, it is appreciated that the systems and methods of the present invention may be applied to kinematic evaluation of other anatomical features of the body, e.g. shoulder, elbow, hip, ankle, spinal segments, etc.

Another aspect of the present invention is an activity recognition system and method that uses gyroscope and accelerometer data to quantify knee instability during a pivot shift event. The system and method of the present invention computes a number of metrics that closely correlate measurements with a clinical grade, and, based on advanced statistical computing methods, a quantified measure of knee stability is computed.

Another aspect is a system and method of computing knee angle using AHRS algorithms to indirectly measure the kinematics of the knee during a pivot shift examination. Data relating to rotation of the tibia relative to the femur is evaluated to assess the state of the patient's ACL as a function of the flexion curve.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic diagram that shows an exemplary sensing device in accordance with the present invention.

FIG. 2 is a schematic diagram that illustrates a pair of sensing devices shown in FIG. 1 attached to the lower leg and upper leg in a configuration for kinematic evaluation of the knee of a patient.

FIG. 3 is a schematic reference diagram of components of the human upper leg and lower leg with respect to the knee of a patient.

FIG. 4 is a schematic flow diagram that illustrates a system for kinematic evaluation and classification of motion characteristics in accordance with the present invention.

FIG. 5 illustrates a flow diagram of the primary components of the kinematic evaluation and classification software of the present invention.

FIG. 6 is a schematic flow diagram of a knee kinematic evaluation and classification method of the present invention.

FIG. 7 is a plot of data from one such pivot shift examination in an ACL-deficient knee, in particular: rotational velocity, directly measured rotation angle, and integrated rotation angle of the tibia during a representative pivot shift event.

FIG. 8 shows a comparison of tibial rotation as found using the gyroscope device and through direct ground measurement.

FIG. 9 shows the change in tibial rotation during pivot shift events due to detaching the ACL.

### DETAILED DESCRIPTION OF THE INVENTION

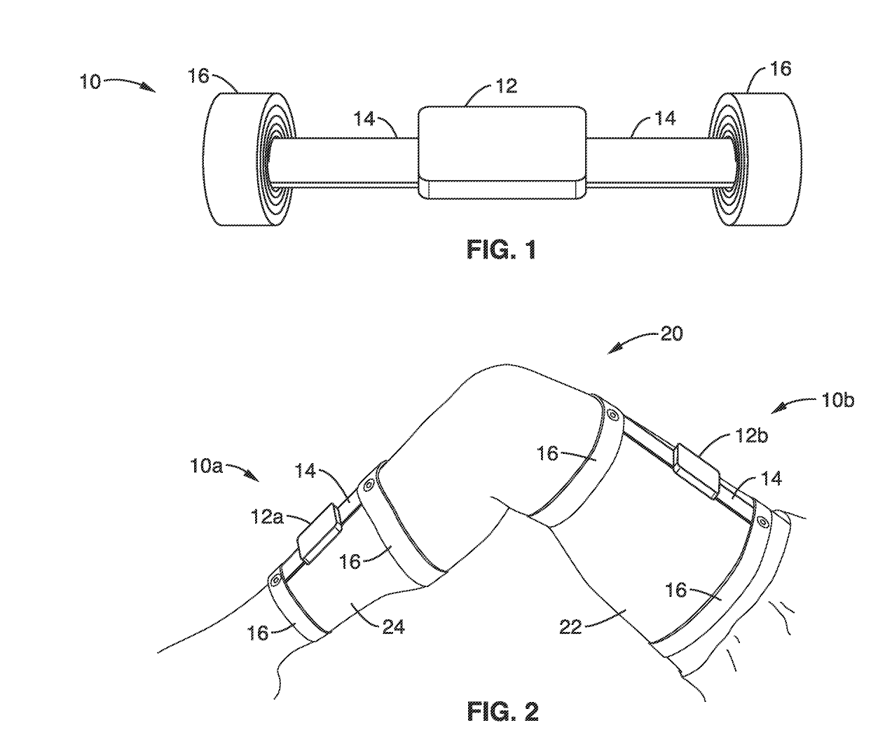

FIG. 1 shows an exemplary sensing device 10 in accordance with the present invention. Sensing device 10 comprises a sensor unit 12 that is attached to support member 14, and a pair of straps 16 (shown in a coiled configuration) disposed at the ends of the support member 14 for coupling the sensing device 10 to a target anatomy of the patient.

FIG. 2 illustrates a pair of sensing devices 10a and 10b attached to the lower leg 24 and upper leg 22, respectively, in a configuration for kinematic evaluation of the knee 20 of a patient. Sensing devices 10a and 10b each comprise respective sensor units 12a and 12b for obtaining measurements corresponding to the orientation of the lower leg 24 and upper leg 22 during specified motions.

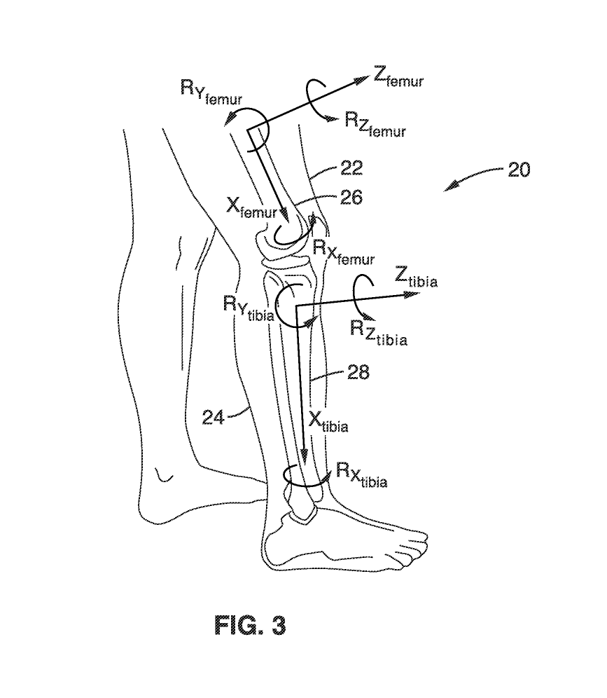

FIG. 3 illustrates a reference diagram of components of the human upper leg 22 and lower leg 24 with respect to knee 20, along with coordinate axes (X_{femur}, Y_{femur}, Z_{femur}) and (X_{tibia}, Y_{tibia}, Z_{tibia}) based on the femur 26 and tibia 28. According to this reference system, X_{femur} lies along the axis of the femur 26, with Z_{femur} oriented orthogonally anterior with respect to X_{femur} and Y_{femur} orthogonally medial (into the page in FIG. 3) to X_{femur}. Correspondingly, X_{tibia} lies along the axis of the tibia 28, with Z_{tibia} oriented orthogonally anterior with respect to X_{tibia} and Y_{tibia} orthogonally medial (into the page in FIG. 3) to X_{tibia}.

In a preferred embodiment, the sensing devices 10a and 10b are configured to be attached to the lower leg 24 and upper leg 22 such that the sensor axes line up with the intended axes of measurement. In particular, the support member 14 of sensing device 10a is substantially parallel to the tibia 28 (X_{tibia}) and the support member 14 of sensing device 10b is substantially parallel to the femur 26 (X_{femur}). As will be described in further detail below, sensing devices are configured to get acceleration measurements in coordinate axes (X_{femur}, Y_{femur}, Z_{femur}) and (X_{tibia}, Y_{tibia}, Z_{tibia}), and rotation measurements in (R_{X-femur}, R_{Y-femur}, R_{Z-femur}) and (R_{X-tibia}, R_{Y-tibia}, R_{Z-tibia}).

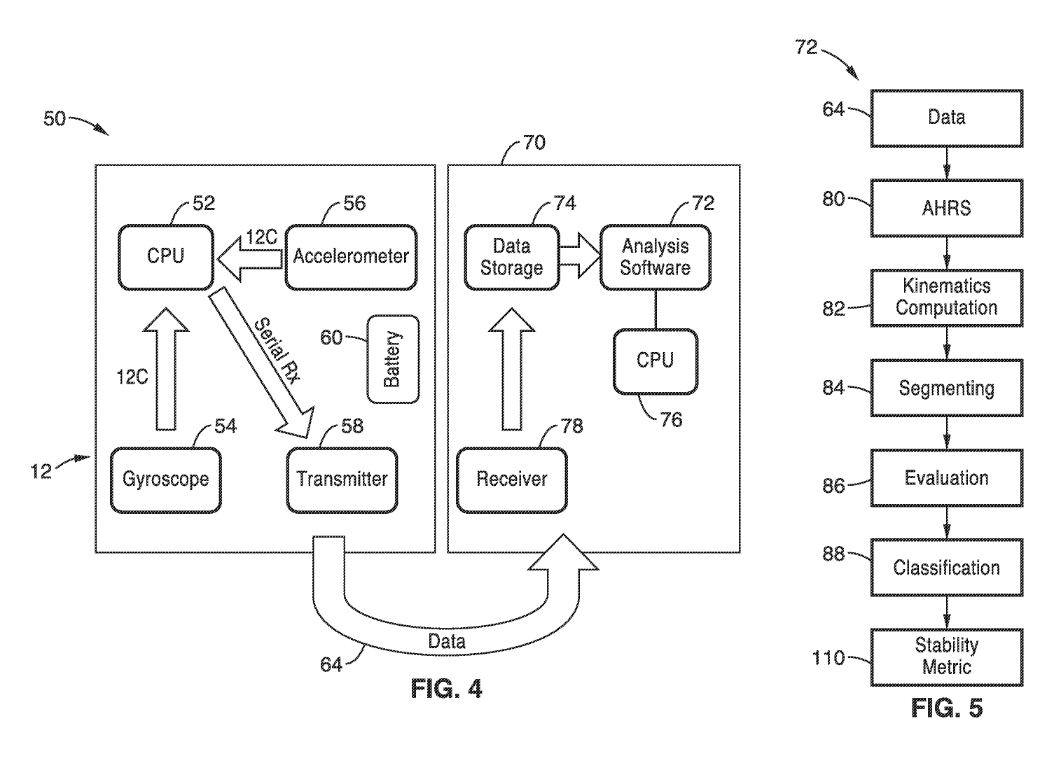

FIG. 4 illustrates a system 50 for kinematic evaluation and classification of motion characteristics in accordance with the present invention. System 50 includes at least one sensor unit 12, and a backend computing device 70 for analyzing output from the sensor unit(s) 10. In a preferred embodiment, the system 50 device is configured to aid clinicians in non-invasively measuring dynamic and kinematic characteristics of the knee to quantify dynamic knee laxity.

The sensor unit 10 preferably includes a three Degree-of-Freedom (3-DOF) gyroscope 54 capable of measuring rotational rates (e.g. up to 2000 deg/sec or more at sample rates of up to 8000 Hz or better). Also included in the sensor unit 10 is a 3-DOF accelerometer 56 capable of measuring accelerations (e.g. up to 16 g or more at sample rates of 3200 Hz or better). In one embodiment, the footprint of the sensor unit is approximately 4×4×0.9 mm.

In one exemplary embodiment, the gyroscope sensor 54 comprises an ITG-3200 gyroscope from Invensense. The ITG-3200 is a Micoelectromechanical Systems (MEMS) vibrating structure gyroscope. In such a sensor, resonant vibrations are stimulated in a mass. Because the vibrating object tends to continue vibrating in the same plane, the mass resists rotation, causing a Coriolis force in response to rotation of the sensor. This force is measured using a transducer and subsequently sampled using a 16-bit Analog to Digital Converter (ADC). The maximum speed measurable using the ITG-3200 is 2000 degrees per second, and it is capable of sample rates up to 8000 Hz.

In one exemplary embodiment, the accelerometer 56 comprises an ADXL345 accelerometer produced by Analog Devices. In this configuration, a transducer measures acceleration. This measurement is digitized via ADC and can be retrieved from the ADXL345 using either an SPI or I2C interface. The device is capable of measuring accelerations up 16 g at sample rates of up to 3200 Hz.

In one exemplary embodiment, the gyroscope 54 and accelerometer 56 are sampled via the I2C communication protocol by an ATMega328p, 8-bit AVR microcontroller 52 produced by Atmel.

A battery 60, or other portable power source, may be coupled to and power the components of sensor unit 12. In one embodiment, battery 60 is connected by means of an external connector (not shown). When battery 60 is connected, the device powers on, and, after a brief startup routine, begins to poll its sensors 54, 56.

Each time the sensors 54, 56 are polled, the resulting data (e.g. data packets 64) are time-stamped and transmitted wirelessly via the Bluetooth transmitter 58. The transmitted data 64 is received by the backend device 70, which includes a receiver 78 (e.g. Bluetooth) and stored within memory 74 for further analysis. Backend device 70 includes analysis software 72 executable on processor 76 for performing kinematic evaluation and classification based on the acquired data 70.

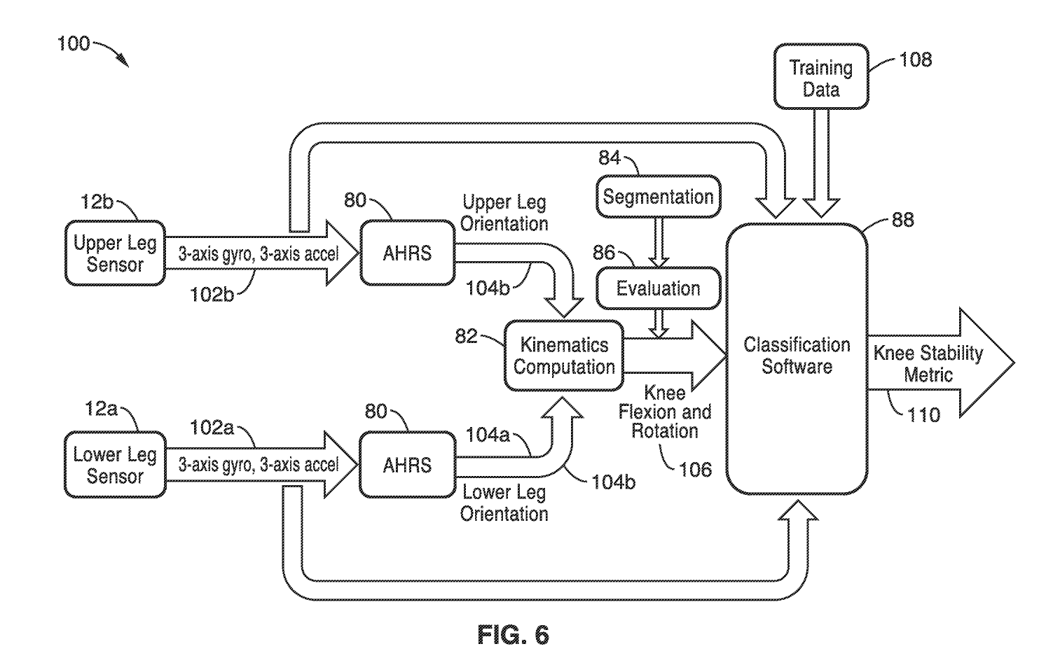

FIG. 5 illustrates a high-level flow diagram of the primary components of the kinematic evaluation and classification software 72 of the present invention. A number of features measured directly using data 64 acquired from the accelerometers 56 and gyroscopes 54 are of critical importance in evaluating knee dynamics. In addition, the absolute angles of the femur 26 and tibia 28 are of interest for a number of reasons. FIG. 6 illustrates a flow diagram of a knee kinematic evaluation and classification method 100 of the present invention, which employs the individual modules shown in FIG. 5.

These angular orientations are computed from 3-axis gyroscope and accelerometer data 102a and 102b (components of transmitted data 64) using algorithms provided in Altitude and Heading Reference Systems (AHRS) module 80. Module 80 takes the gyroscope 54 outputs within transmitted data 64, which scale linearly with rotation rates, and integrates them to compute the three angles that specify the target objects orientation (e.g. (R_{X-femur}, R_{Y-femur}, R_{Z-femur}) for femur 26 and (R_{X-tibia}, R_{Y-tibia} R_{Z-tibia}) for tibia 28). However, due to sensor inaccuracy, ADC round-off, and numerical error, this integration eventually accrues significant error. To mitigate this effect, measurements from long-term stable accelerometers 56 are used to correct this long term error.

Accelerometers 56 are generally only useful to measure pitch and roll angles during periods characterized by zero acceleration, as changes in velocity confound the use of accelerometers to measure the gravity vector. Because accelerations generally only occur for a limited period of time, the AHRS module applies low-pass filtering to reduce the effects of such accelerations, and the resulting signals from accelerometers 56 are then used to gently correct errors from integration of gyroscope 54 signals. Thus, AHRS module 80 uses gyroscope 54 signals to responsively compute orientation during rotations and accelerometer 56 data to provide long-term accuracy.

Application of the two sensing devices 10a and 10b along the tibia 28 and femur 26 enables kinematic and dynamic evaluation of the upper leg 24 and lower leg 22, and thereby, of the knee 20. By computing the difference between the absolute orientations 104a and 104b of the lower leg 24 and upper leg 22, the angles of rotation in the knee 20 can be determined, which is of particular interest in evaluating knee stability.

The output lower and upper leg orientation data 104a and 104b from AHRS module 80 data is then used to compute knee flexion and rotation via the kinematics computation module 82.

Angular data from 104a and 104b outputted by AHRS module 80 is contained in a 3×3 rotation matrix, R, which describes the orientation of the sensor units 12a and 12b. From sensor data 102a and 102b, the AHRS module computes R_{femur} (R_{X-femur}, R_{Y-femur}, R_{Z-femur}) and R_{tibia} (R_{X-tibia}, R_{Y-tibia}, R_{Z-tibia}).

From this data, kinematics computation module 82 computes Eq. 1:

RₖₙₑₑR_{femur}=R_{tibia}, (Eq. 1)

where Rₖₙₑₑ is a rotation matrix representing the set of rotations through the knee.

This yields Eq. 2:

Rₖₙₑₑ=R_{tibia}Rₖₙₑₑ⁻¹. (Eq. 2)

The output of Rₖₙₑₑ 106 comprises articulation components for both the flexion angle and rotation angle of knee 20.

The flexion angle (primarily components of the tibia 26 and fibula 28 in the Y-axis) is of significant importance, as it enables autonomous detection of pivot shift events that are calculated in segmentation module 84 and evaluation module 86. In particular, the start and endpoints of such pivot shift events are accurately determined in the segmentation module 84, thereby enabling autonomous characterization of the knee.

Next, evaluation module 86 evaluates whether a pivot shift event has occurred based on the segmented data from the start and end points. Evaluation module 86 is configured to detect an unsuccessful pivot shift examination, and remove it from consideration.

The pivot shift test, of which a pivot shift event is the desired action, is a clinically useful exam to assess the stability of knees that have sustained ACL injury. It can often be difficult for a physician to reliably illicit the rotational instability associated with ACL deficiency using a pivot shift examination. Such instability is uncomfortable for patients, and they may “guard” against it by tensing muscles surrounding the knee. As a result, some attempts at pivot shift events are unsuccessful, whereas subsequent efforts might be more successful in eliciting instability. Without automated pivot event detection, a clinician is required to manually denote start and endpoints via visual inspection, which is a time-intensive and unreliable procedure which requires significant training. Thus, autonomous detection of pivot shift events is of paramount importance for a clinically viable system.

In the pivot shift test, a valgus moment is applied to the tibia (sometimes in combination with a slight internal tibial torque), causing an internal rotation of the tibia and a corresponding anterior subluxation of the lateral tibial plateau near full extension. A pivot shift event generally comprises a range of knee articulation involving a starting flexion point in one rotational direction to a point of furthest flexion, and then back to the starting point. As the examiner flexes the knee from 0 to 90°, the line of action of the iliotibial band changes, causing it to become a knee flexor. The resulting external tibial torque causes the tibia to rotate externally, which spontaneously reduces the anteriorly subluxated lateral tibial plateau, producing a pivot shift. While the pivot shift test is widely accepted as a measure of rotational stability, there are currently no objective means of quantifying this exam.

Next, the segmented knee flexion and rotation data 106, along with raw 3-axis data 102a and 102b from the lower and upper sensor units 12a and 12b, are input into a classification module 88. The classification module 88 is configured to apply training data 108 to weigh aspects of the input data 102a, 102b and 106 to generate one or more metrics 110 relating to the kinematics of the target anatomy.

In a preferred embodiment, classification module is configured to generate a clinical grade (e.g. classification of 1, 2 or 3) relating to the stability of the knee, and in particular the ACL. The training data is preferably built using the system 50 sensing devices 12a and 12b on a number of patients through a plurality of kinematic motions/measurements, and stored in a training data database 108. Specific features of the acquired sensor data are highly correlated with knee stability. In particular, accelerations in the anterior or z-axis direction on the tibia, and rotation of the tibia relative to the femur, are significant indicators of knee stability, and are highly weighted in classification module 88 to generate the stability metric 110.

While the two-sensor device (e.g. sensing devices 10a and 10b) configuration shown in FIG. 2 is a preferred method of the present invention, it is contemplated that kinematic evaluation of joints may be performed using joint data from only a single device (e.g. sensing device 10a affixed along only one of the body members (e.g. the lower leg 24/tibia 28), particularly once training data 108 is acquired.

Experimental Results

Preliminary tests were conducted on the sensor device 10 of the present invention. The rate sensor (gyroscope 54) was calibrated by repeatedly rotating the measurement device through an arc of 63 degrees and comparing the integrated result to a direct measurement of the rotation established by a ground sensor. Thirty such rotations were performed at speeds representative of those of tibial rotations during pivot shifts, and a least squares method was used to compute the linear constant relating the measured rotations to the result of the integration of rotation rate. After calibration, the average error between the two results was 0.07 degrees with a standard deviation of 1.2 degrees.

A series of manual pivot shifts were then performed on a total of 9 cadaveric knees with the ACL intact and subsequently removed using a dummy load cell. Ten pivot shifts were completed for each knee state, and the tibial rotation, as determined via direct measurement and integration of rotational rate was recorded for each repetition.

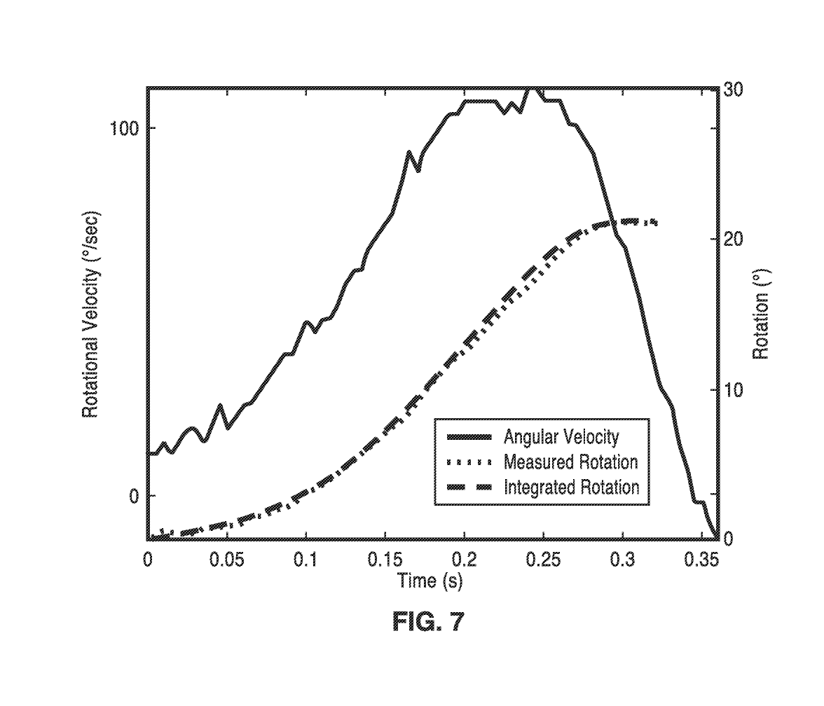

FIG. 7 is a plot of data from one such pivot shift examination in an ACL-deficient knee, in particular: rotational velocity, directly measured rotation angle, and integrated rotation angle of the tibia during a representative pivot shift event. This knee is in the ACL deficient state and reaches a rotational velocity of roughly 105°/sec while traversing an arc of roughly 21 degrees.

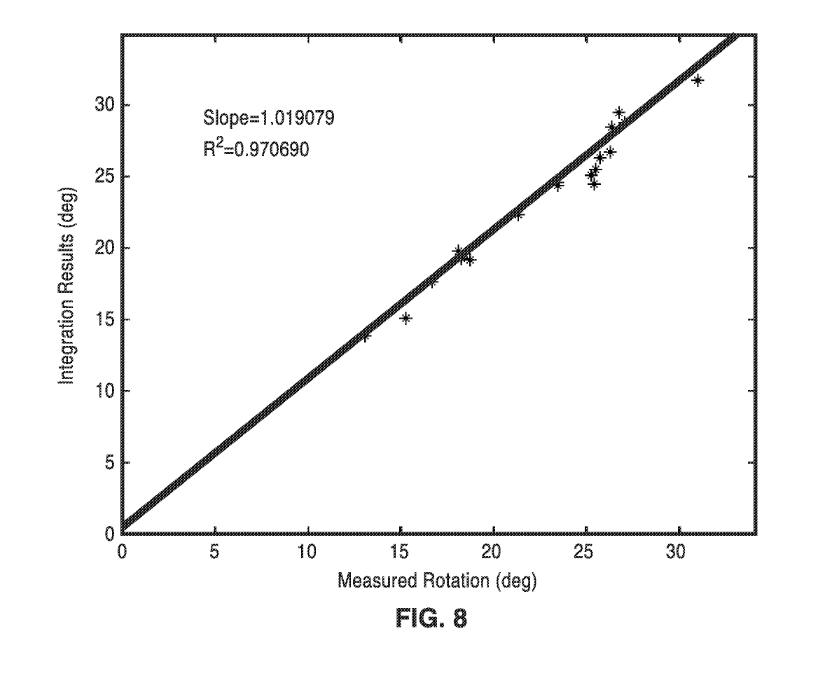

In order to demonstrate the accuracy of the proposed gyroscope, the results of direct measurement for all knees and all knee conditions are compared to those found using the gyroscope device. These results are plotted against each other in FIG. 8, which shows a comparison of tibial rotation as found using the gyroscope device and through direct ground measurement. This clearly demonstrates excellent agreement between the two forms of measurement, as supported by the very high R-squared coefficient and the near unity slope of the line of best fit. This data indicates that measuring tibial rotation by integrating angular velocity data yields accurate results, thereby validating the proposed use of a gyroscope for this purpose.

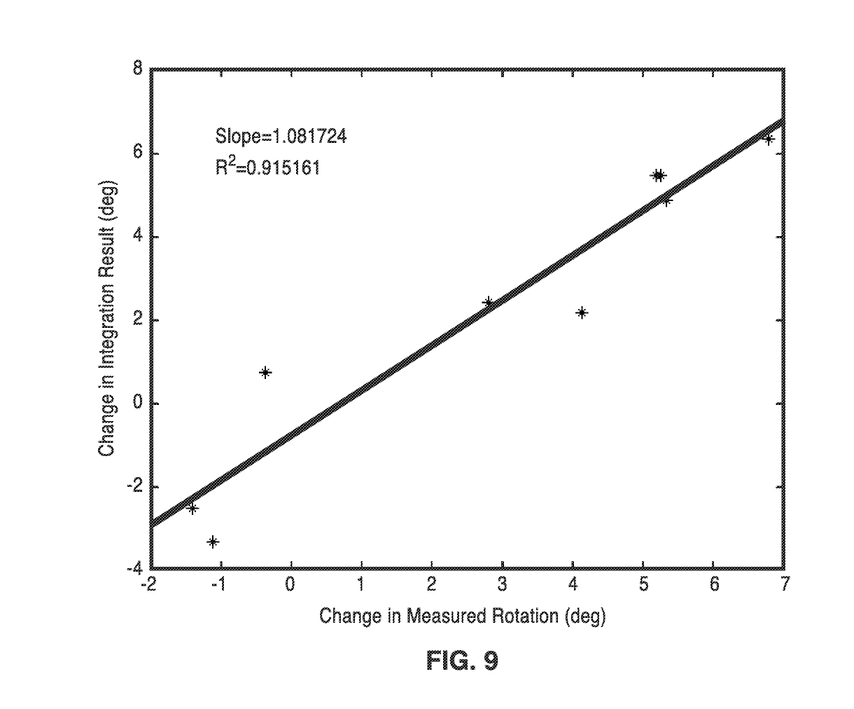

Another objective of preliminary experiments was to determine the utility of the gyroscope 54 in determining rotational differences between ACL-intact and ACL-deficient knees during the pivot shift maneuver. To this end, we measured tibial rotation during the pivot shift maneuver in the ACL-deficient and ACL-intact knee, and measured change in rotation as found using integration of gyroscope data as plotted against direct measurements in FIG. 9, which shows the change in tibial rotation during pivot shift events due to detaching the ACL. Results from the gyroscope 54 are shown compared to directly measured results.

The corresponding data is shown in Table 1. A strong agreement is again evident, indicating that gyroscope devices can be reliable in discerning differences in tibial rotation between the ACL-intact and ACL-deficient state during the pivot shift test as measured by the gyro and compared to ground measurements. Of the 9 knees considered in this study, direct measurements show that 6 of the 9 (bold in Table 1) demonstrate statistically significant increases in tibial rotation in response to sectioning of the ACL.

The system and methods of the present invention may be utilized by clinicians in at least the following ways: 1) to evaluate the magnitude of knee ligament laxity following injury, 2) to evaluate the efficacy of ligament reconstruction following surgery, and 3) to study knee kinematics following ligament injury. It is also contemplated that the systems and methods of the present invention may be used in evaluating the stability of other ligaments within the knee (e.g. the lateral and medial collateral ligaments or posterior cruicate ligament), or to evalutate to pattern complex motion around other joints, including the shoulder, elbow, wrist, hip and ankle.

The device has a number of potential commerical applications including 1) the evaluation of pre- and post-operative knee stability, both in the clinic and operating room, 2) use as a research tool for assessing multiplanar knee stability, and 3) assessing injury mechanisms, specifically the potential origins of ACL injury.

Furthermore, while stability is one metric that may be evaluated using the systems of the present invention, it is contemplated that the system and method of the present invention may also be implemented to evaluate other physiological/kinematic characteristic of skeletal joints, e.g. range of motion, acceleration, etc. For example, the sensing units may be applied to particular body members, (e.g. hand, lower arm, upper arm, torso, foot, etc.), and used in conjunction with specific training data, to evaluate a variety of motions involved with medical treatment, or evaluation of an individual's athletic attributes (e.g. golf or baseball swing, pitching motion, running stride, kick, etc.).

The system and methods of the present invention have a number of potential advantages. Foremost, the system and methods of the present invention allow for one to measure both rotational and linear moments about the knee. Currently, there is no device that performs this function that can be utilized in the clinical or research setting. Secondly, the system and method of the present invention is both small and non-invasive, which allows it to be used without causing discomfort to the patient and can be applied in a variety of settings including the clinic, operating room, training room, sidelines, etc. It does not need to be invasively attached to the patient. Thirdly, the system and methods of the present invention allows for accurate conversion of rotational velocity into absolute angles. Accordingly, in addition to information about patterns of acceleration and velocity change, our device produces objective quantification of rotational change. This value is of greater significance to those individuals studying knee kinematics and knee injury and is more easily interpreted and compared to contemporary measurements of knee instability.

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

From the discussion above it will be appreciated that the invention can be embodied in various ways, including the following:

1. A system for kinematic evaluation of a skeletal joint having at least one body member, comprising: a sensor unit comprising an accelerometer and a gyroscope; wherein the sensor unit is configured to attach to a first body member of the skeletal joint to acquire data with respect to the first body member; wherein said data comprises acceleration data from the accelerometer and rotation data from the gyroscope; a processor coupled to the sensor unit; and programming executable on the processor for: computing orientation data relating to the first body member from one or more of the acquired acceleration data and rotation data; and generating one or more metrics from the orientation data; the one or more metrics relating to a kinematic characteristic of the skeletal joint.

2. A system as in any of the previous embodiments: wherein the programming comprises an Altitude and Heading Reference System (AHRS) module for computing the orientation data; and wherein computing the orientation data comprises: integrating the rotation data from the gyroscope; and applying the acceleration data to correct for long term error associated with the integrated rotation data.

3. A system as in any of the previous embodiments, wherein the sensor unit comprises a first sensor unit comprising a first accelerometer and a first gyroscope, and the skeletal joint further comprises a second body member, the system further comprising:a second sensor unit comprising a second accelerometer and a second gyroscope; wherein the second sensor unit is configured to attach to the second body member of the skeletal joint to acquire data with respect to the second body member; wherein said second body member data comprises acceleration data from the second accelerometer and rotation data from the second gyroscope; and wherein the programming is further configured for computing orientation data relating to the second body member.

4. A system as in any of the previous embodiments: wherein skeletal joint comprises a knee; wherein the first body member comprises an upper leg and the second body member comprises a lower leg: and wherein the programming is further configured for: computing knee rotation angle data and knee flexion angle data from the computed orientation data.

5. A system as in any of the previous embodiments, wherein the kinematic characteristic comprises an indication of knee stability.

6. A system as in any of the previous embodiments, wherein the one or more metrics comprise a clinical grade relating to the knee.

7. A system as in any of the previous embodiments, the programming further configured for autonomously evaluating a pivot shift event associated with the knee as a function of the computed knee flexion angle.

8. A system as in any of the previous embodiments, wherein the programming is further configured for detecting a starting point and ending point of the pivot shift event.

9. A system as in any of the previous embodiments, wherein the programming is further configured for: applying weights to the acceleration data, rotation data, knee rotation angle data and knee flexion angle data to generate said one or more metrics.

10. A system as in any of the previous embodiments, wherein the weights are determined according to training data acquired from the first sensor unit and the second sensor unit.

11. A system for kinematic evaluation of a skeletal joint having at least one body member, comprising: a processor; and programming executable on the processor for: acquiring data relating to a first body member of the skeletal joint from a sensor unit comprising an accelerometer and a gyroscope; wherein said data comprises acceleration data from the accelerometer and rotation data from the gyroscope; computing orientation data relating to the first body member from one or more of the acquired acceleration data and rotation data; and generating one or more metrics from the orientation data; the one or more metrics relating to a kinematic characteristic of the skeletal joint.

12. A system as in any of the previous embodiments: wherein computing the orientation data comprises: integrating the rotation data from the gyroscope; and applying the acceleration data to correct for long term error associated with the integrated rotation data.

13. A system as in any of the previous embodiments, wherein the sensor unit comprises a first sensor unit comprising a first accelerometer and a first gyroscope, and the skeletal joint further comprises a second body member, the programming further configured for: acquiring second body member data relating to a second body member of the skeletal joint from a second sensor unit comprising a second accelerometer and a second gyroscope; and computing orientation data relating to the second body member.

14. A system as in any of the previous embodiments: wherein the skeletal joint comprises a knee; wherein the first body member comprises an upper leg and the second body member comprises a lower leg: and wherein the programming is further configured for: computing knee rotation angle data and knee flexion angle data from the computed orientation data.

15. A system as in any of the previous embodiments, wherein the kinematic characteristic comprises an indication of knee stability.

16. A system as in any of the previous embodiments, wherein the one or more metrics comprise a clinical grade relating to the knee.

17. A system as in any of the previous embodiments, the programming further configured for autonomously evaluating a pivot shift event associated with the knee as a function of the computed knee flexion angle.

18. A system as in any of the previous embodiments, wherein the programming is further configured for detecting a starting point and ending point of the pivot shift event.

19. A system as in any of the previous embodiments, wherein the programming is further configured for: applying weights to the acceleration data, rotation data, knee rotation angle data and knee flexion angle data to generate said one or more metrics.

20. A system as in any of the previous embodiments, wherein the weights are determined according to training data acquired from the first sensor unit and the second sensor unit.

21. A method for kinematic evaluation of a skeletal joint having at least one body member, comprising: acquiring data relating to a first body member of the skeletal joint from a sensor unit comprising an accelerometer and a gyroscope; wherein said data comprises acceleration data from the accelerometer and rotation data from the gyroscope; computing orientation data relating to the first body member from one or more of the acquired acceleration data and rotation data; and generating one or more metrics from the orientation data; the one or more metrics relating to a kinematic characteristic of the skeletal joint.

22. A method as in any of the previous embodiments: wherein computing the orientation data comprises: integrating the rotation data from the gyroscope; and applying the acceleration data to correct for long term error associated with the integrated rotation data.

23. A method as in any of the previous embodiments, wherein the sensor unit comprises a first sensor unit comprising a first accelerometer and a first gyroscope, and the skeletal joint further comprises a second body member, the method further comprising: acquiring second body member data relating to a second body member of the skeletal joint from a second sensor unit comprising a second accelerometer and a second gyroscope; and computing orientation data relating to the second body member.

24. A method as in any of the previous embodiments: wherein the skeletal joint comprises a knee; wherein the first body member comprises an upper leg and the second body member comprises a lower leg: and wherein the method further comprises: computing knee rotation angle data and knee flexion angle data from the computed orientation data.

25. A method as in any of the previous embodiments, wherein the kinematic characteristic comprises an indication of knee stability.

26. A method as in any of the previous embodiments, wherein the one or more metrics comprise a clinical grade relating to the knee.

27. A method as in any of the previous embodiments, further comprising: autonomously evaluating a pivot shift event associated with the knee as a function of the computed knee flexion angle.

28. A method as in any of the previous embodiments, further comprising: detecting a starting point and ending point of the pivot shift event.

29. A method as in any of the previous embodiments, further comprising: applying weights to the acceleration data, rotation data, knee rotation angle data and knee flexion angle data to generate said one or more metrics.

30. A method as in any of the previous embodiments, wherein the weights are determined according to training data acquired from the first sensor unit and the second sensor unit.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Average Tibial Rotations As Found Using Integration
Of Gyroscope And By Direct Measurement

| | Inte- | Inte- | Inte- | | | | | |
|---|---|---|---|---|---|---|---|---|
| | grat- | grat- | grat- | | Mea- | Mea- | Mea- | |
| | ed | ed | ed | T- | sured | sured | sured | T- |
| Knee | ACD | ACL | STD | value | ACD | ACL | STD | value |
| 1 | 19.30 | 13.84 | 2.20 | 0.0005 | 18.27 | 13.08 | 2.22 | 0.0009 |
| 2 | 31.77 | 26.32 | 1.06 | 0.0000 | 31.06 | 25.81 | 0.83 | 0.0000 |
| 3 | 25.50 | 19.16 | 1.06 | 0.0000 | 25.52 | 18.74 | 1.10 | 0.0000 |
| 4 | 24.52 | 22.35 | 1.53 | 0.0448 | 25.48 | 21.35 | 1.10 | 0.0000 |
| 5 | 24.58 | 19.73 | 0.92 | 0.0000 | 23.49 | 18.16 | 0.99 | 0.0000 |
| 6 | 26.77 | 24.36 | 0.89 | 0.0001 | 26.31 | 23.52 | 0.94 | 0.0000 |
| 8 | 29.51 | 28.80 | 1.05 | 0.3395 | 26.79 | 27.15 | 1.05 | 0.1322 |
| 7 | 15.07 | 17.61 | 1.04 | 0.0929 | 15.30 | 16.71 | 0.90 | 0.1232 |
| 9 | 25.09 | 28.45 | 0.86 | 0.1039 | 25.30 | 26.42 | 0.61 | 0.0828 |
| Avg. | 24.68 | 22.29 | 1.18 | | 24.17 | 21.21 | 1.08 | |

What is claimed is:

1. A system for kinematic evaluation of a skeletal joint having at least one body member, comprising:
a sensor unit comprising an accelerometer and a gyroscope;
wherein the sensor unit is configured to attach to a first body member of the skeletal joint to acquire data with respect to the first body member;
wherein said data comprises acceleration data from the accelerometer and rotation data from the gyroscope;
a processor coupled to the sensor unit; and
programming executable on the processor for:
computing orientation data relating to the first body member from one or more of the acquired acceleration data and rotation data; and
generating one or more metrics from the orientation data;
the one or more metrics relating to a kinematic characteristic of the skeletal joint.

2. A system as recited in claim 1:
wherein the programming comprises an Altitude and Heading Reference System (AHRS) module for computing the orientation data; and
wherein computing the orientation data comprises:
integrating the rotation data from the gyroscope; and
applying the acceleration data to correct for long term error associated with the integrated rotation data.

3. A system as recited in claim 2, wherein the sensor unit comprises a first sensor unit comprising a first accelerometer and a first gyroscope, and the skeletal joint further comprises a second body member, the system further comprising:
a second sensor unit comprising a second accelerometer and a second gyroscope;
wherein the second sensor unit is configured to attach to the second body member of the skeletal joint to acquire data with respect to the second body member;
wherein said second body member data comprises acceleration data from the second accelerometer and rotation data from the second gyroscope; and
wherein the programming is further configured for computing orientation data relating to the second body member.

4. A system as recited in claim 3:
wherein skeletal joint comprises a knee;
wherein the first body member comprises an upper leg and the second body member comprises a lower leg: and
wherein the programming is further configured for:
computing knee rotation angle data and knee flexion angle data from the computed orientation data.

5. A system as recited in claim 4, wherein the kinematic characteristic comprises an indication of knee stability.

6. A system as recited in claim 5, wherein the one or more metrics comprise a clinical grade relating to the knee.

7. A system as recited in claim 4, the programming further configured for autonomously evaluating a pivot shift event associated with the knee as a function of the computed knee flexion angle.

8. A system as recited in claim 7, wherein the programming is further configured for detecting a starting point and ending point of the pivot shift event.

9. A system as recited in claim 4, wherein the programming is further configured for:
applying weights to the acceleration data, rotation data, knee rotation angle data and knee flexion angle data to generate said one or more metrics.

10. A system as recited in claim 9, wherein the weights are determined according to training data acquired from the first sensor unit and the second sensor unit.

11. A system for kinematic evaluation of a skeletal joint having at least one body member, comprising:
a processor; and
programming executable on the processor for:
acquiring data relating to a first body member of the skeletal joint from a sensor unit comprising an accelerometer and a gyroscope;
wherein said data comprises acceleration data from the accelerometer and rotation data from the gyroscope;
computing orientation data relating to the first body member from one or more of the acquired acceleration data and rotation data; and
generating one or more metrics from the orientation data;
the one or more metrics relating to a kinematic characteristic of the skeletal joint.

12. A system as recited in claim 11:
wherein computing the orientation data comprises:
integrating the rotation data from the gyroscope; and
applying the acceleration data to correct for long term error associated with the integrated rotation data.

13. A system as recited in claim 12, wherein the sensor unit comprises a first sensor unit comprising a first accelerometer and a first gyroscope, and the skeletal joint further comprises a second body member, the programming further configured for:
acquiring second body member data relating to a second body member of the skeletal joint from a second sensor unit comprising a second accelerometer and a second gyroscope; and
computing orientation data relating to the second body member.

14. A system as recited in claim 13:
wherein the skeletal joint comprises a knee;
wherein the first body member comprises an upper leg and the second body member comprises a lower leg: and
wherein the programming is further configured for:
computing knee rotation angle data and knee flexion angle data from the computed orientation data.

15. A system as recited in claim 14, wherein the kinematic characteristic comprises an indication of knee stability.

16. A system as recited in claim 15, wherein the one or more metrics comprise a clinical grade relating to the knee.

17. A system as recited in claim 14, wherein the programming is further configured for autonomously evaluating a pivot shift event associated with the knee as a function of the computed knee flexion angle.

18. A system as recited in claim 17, wherein the programming is further configured for detecting a starting point and ending point of the pivot shift event.

19. A system as recited in claim 14, wherein the programming is further configured for:
applying weights to the acceleration data, rotation data, knee rotation angle data and knee flexion angle data to generate said one or more metrics.

20. A system as recited in claim 19, wherein the weights are determined according to training data acquired from the first sensor unit and the second sensor unit.

21. A method for kinematic evaluation of a skeletal joint having at least one body member, comprising:
acquiring data relating to a first body member of the skeletal joint from a sensor unit comprising an accelerometer and a gyroscope;
wherein said data comprises acceleration data from the accelerometer and rotation data from the gyroscope;
computing orientation data relating to the first body member from one or more of the acquired acceleration data and rotation data; and
generating one or more metrics from the orientation data;
the one or more metrics relating to a kinematic characteristic of the skeletal joint.

22. A method as recited in claim 21:
wherein computing the orientation data comprises:
integrating the rotation data from the gyroscope; and
applying the acceleration data to correct for long term error associated with the integrated rotation data.

23. A method as recited in claim 22, wherein the sensor unit comprises a first sensor unit comprising a first accelerometer and a first gyroscope, and the skeletal joint further comprises a second body member, the method further comprising:
acquiring second body member data relating to a second body member of the skeletal joint from a second sensor unit comprising a second accelerometer and a second gyroscope; and
computing orientation data relating to the second body member.

24. A method as recited in claim 23:
wherein the skeletal joint comprises a knee;
wherein the first body member comprises an upper leg and the second body member comprises a lower leg: and
wherein the method further comprises:
computing knee rotation angle data and knee flexion angle data from the computed orientation data.

25. A method as recited in claim 24, wherein the kinematic characteristic comprises an indication of knee stability.

26. A method as recited in claim 25, wherein the one or more metrics comprise a clinical grade relating to the knee.

27. A method as recited in claim 24, further comprising:
autonomously evaluating a pivot shift event associated with the knee as a function of the computed knee flexion angle.

28. A method as recited in claim 27, further comprising:
detecting a starting point and ending point of the pivot shift event.

29. A method as recited in claim 24, further comprising:
applying weights to the acceleration data, rotation data, knee rotation angle data and knee flexion angle data to generate said one or more metrics.

30. A method as recited in claim 29, wherein the weights are determined according to training data acquired from the first sensor unit and the second sensor unit.
